(19) United States
(12) Patent Application Publication
(10) Pub. No.: US 2025/0073084 A1
(43) Pub. Date: Mar. 6, 2025

(54) WOUND COVER SYSTEM

(71) Applicants: LOHMANN GMBH & CO. KG, Neuwied (DE); RHEINISCH-WESTFALISCHE TECHNISCHE HOCHSCHULE (RWTH) AACHEN, Aachen (DE); DOCS IN CLOUDS TELECARE GMBH, Aachen (DE)

(72) Inventors: Volker LUTZ, Neuwied (DE); Maike UNKELBACH, Andernach (DE); Michael CZAPLIK, Herzogenrath (DE); Janosch KUNCZIK, Roetgen (DE)

(21) Appl. No.: 18/727,079

(22) PCT Filed: Dec. 19, 2022
(86) PCT No.: PCT/EP2022/086809

§ 371 Date: Jul. 6, 2024

(30) Foreign Application Priority Data
Jan. 7, 2022 (DE) 10 2022 000 063.6

(51) Int. Cl. A61F 13/00; A61B 5/00; A61F 13/02

(52) U.S. Cl. CPC A61F 13/00051; A61B 5/445; A61B 5/6802; A61F 13/02

(57) ABSTRACT

The invention relates to a wound cover system with at least one wound cover comprising a multitude of sensors which are spaced apart and configured to measure at least one wound parameter. In order to improve a wound monitoring and a patient comfort, it is proposed that the wound cover system comprises a control unit which detects local changes of the wound parameter based on the data provided by the sensors

### CROSS-REFERENCE TO RELATED APPLICATIONS

The present application claims priority to German patent application 10 2022 000 063.6, “Wundabdecksystem” (Wound cover system), filed Jan. 7, 2022, which is hereby incorporated by reference.

### FIELD OF TECHNOLOGY

The present technology relates to a wound cover system.

### BACKGROUND

It is known to cover wounds, especially wounds remaining after an operation, with a wound cover, wherein the wound cover usually comprises adhesive areas at the edges to prevent an infection of the wound due to outside contact. This, however, bears the significant disadvantage of fully covering the wound, requiring a laborious and frequent detaching of the wound cover to detect and treat any infections or inflammations that occur despite the presence of the wound cover. This puts stress on both the patient and the medical staff and may, in the worst case, lead to a re-opening and/or a contamination of the wound. Therefore, there is a need for a precise and constant wound monitoring which does not require a detaching of the wound cover and does not negatively affect a comfort of the patient even during long-term usage.

It is an object of the present technology to provide a wound cover system with advantageous properties concerning a wound monitoring and a patient comfort. This object is achieved by the features of claims 1 and 14, while other embodiments and improvements of the present technology may be taken from the dependent claims.

### BRIED SUMMARY

The present technology relates to a wound cover system with at least one wound cover comprising a multitude of sensors which are spaced apart and configured to measure at least one wound parameter.

### DESCRIPTION

It is proposed that the wound cover system further comprises a control unit which detects local changes of the wound parameter based on the data provided by the sensors. This way, a precise wound monitoring may be achieved. Advantageously, dangerous changes of the wound parameter indicating an injury and/or infection and/or inflammation of the wound can be detected promptly without requiring a detaching of the wound cover. Especially advantageously, the data may not only provide information about the general state of the wound but may also be used in case of an injury and/or infection and/or inflammation to determine the area of the wound in which the injury and/or infection and/or inflammation is located. This is especially helpful with large and/or deep wounds, which commonly remain after an operation, allowing for a prompt and efficient treatment.

The wound cover may be a wound dressing or a wound plaster. Furthermore, the wound cover may be a cover configured to reduce bleeding or a compression cover. Preferably, the wound cover comprises at least one cover material into which the sensors are embedded. The cover material is preferably configured to cover the wound and, especially preferably, absorb fluids exuded from the wound. Alternatively or additionally, the cover material may be configured to prevent a movement of the wound. The cover material may, depending on the application, comprise any materials known to one skilled in the art, such as metal, plaster, wood, plastic, textiles such as cotton, foams, alginates, hydrocolloids, gels, gauzes and/or cellulose.

Each of the sensors may be configured to measure exactly one wound parameter. Preferably, at least one of the sensors is configured to measure a multitude of wound parameters. The sensors may be different to each other, preferably, the sensors are identical to each other. The wound parameter may be a pH, a temperature, a moisture or a pressure. The wound cover may comprise any number of sensors which may be arranged in any way. For example, the sensors may be arranged in a line or a matrix. Alternatively, the sensors may be arranged randomly, or form the shape of a geometrical figure such as a triangle, a rectangle or especially an oval. In a top view of the wound cover, the sensors may not touch or overlap each other. Advantageously, the sensors each define a sensor area in which the sensor achieves a sufficiently precise measurement of the wound parameter. Especially advantageously, the sensors are spaced in a way so that the sensor areas form a coherent greater sensor area with only minimal overlap of the individual sensor areas.

The sensors may be directly connected to the cover material. For example, the sensors may be sewn, welded, clamped and/or adhered to the cover material. Furthermore, the sensors may be SMD components such as chips. The sensors may be part of a sensor array, wherein the sensors may be connected to a mutual interface via convergent tracks, the mutual interface assisting in forwarding the data to the control unit. Alternatively, at least one of the sensors may be arranged separately to the others and may be provided with its own interface.

The control unit preferably comprises at least one processor for processing and analyzing the data. The control unit may use a multitude of wound parameters to determine a specific value for evaluating a state of the wound. The specific value could be displayed as a number and/or a color. This way, the patient or medical staff can be informed about the state of the wound in a prompt and reliable manner. The control unit may be arranged at the wound cover, for example, the control unit may be embedded into the cover material or arranged on a side of the wound cover opposite of the wound.

The wound cover may not comprise any adhesive areas and may be, for example, fixed over the wound via a wrap, a knot and/or a separate adhesive strip. In order to achieve an efficient wound healing and reduce labor of medical staff, it is proposed that the wound cover comprises an adhesive area and a cover area, wherein the sensors are distributed over the cover area. Advantageously, the adhesive area forms a closed edge around the cover area. In the top view of the wound cover, when separating the cover area into four quadrants, at least one sensor may be arranged in each of the quadrants. The greater sensor area may extend over less than 60% of the cover area. For example, the greater sensor area may be formed by a multitude of separate sensor areas, which are distributed over the cover area. Preferably, the greater sensor area extends over at least 60%, especially preferably at least 75% of the cover area. Especially preferably, the greater sensor area is a coherent area. This way, a precise and constant wound monitoring can be achieved without requiring a frequent detaching of the wound cover. Advantageously, infections of the wound due to outside contact and a re-opening of the wound due to frequent detaching of the wound cover can be prevented. Especially advantageously, the entire wound area can be efficiently monitored by the sensors.

The sensors may be distributed over the cover area in any way. To achieve an especially efficient monitoring of the wound area, it is proposed that the sensors are distributed over a partial area of the cover area, wherein the partial area forms an oval shape in the top view of the wound cover. The partial area may form a circular shape in the top view of the wound cover. Preferably, the partial area forms an elongated oval shape in the top view of the wound cover. For example, the sensors may be arranged along an edge of the partial area, alternatively, the sensors may be arranged along a longitudinal axis of the partial area. Advantageously, the partial area encompasses at least 60%, especially advantageously at least 75% of the cover area. This way, a natural tendency of wounds to form oval shapes can be exploited to monitor the entirety of the wound area via the sensors more efficiently.

The sensors may be arranged on a rigid circuit board. To achieve a stable construction, which is suitable for skin applications, it is proposed that the wound cover comprises a substrate carrying the sensors, wherein the substrate and the sensors together form a flexible unit. The substrate may comprise any materials known to one skilled in the art, such as plastics, ceramics, glass and/or cellulose. Preferably, the substrate is a flexible foil. Preferably, in an application state, the flexible unit adapts to a shape of a surface covered by the flexible unit. Preferably, the flexible unit can endure at least 1000 instances of the covered surface bending by up to 90°, which may for example occur when applying the wound cover on an inside of an elbow of a patient. The flexible unit “enduring” the instances is to be understood as the flexible unit exhibiting no damages or permanent deformations after the instances. Especially preferably, a reset force applied to the flexible unit during any bending of the covered surface is smaller than the adhesive force of the adhesive area. In the case of the sensors being SMD components, the sensors are advantageously arranged in a way that spaces between neighboring sensors are large enough to prevent the sensors from touching each other during a bending of the flexible unit. Especially advantageously, the sensors together cover at most 70% of the substrate. Advantageously, a height of the sensors is at most ten times a height of the substrate. This way, the wound cover may be applied to any skin surface. Advantageously, an involuntary detaching of the wound cover caused by a high reset force being applied to the flexible unit can be prevented.

The sensors may be applied on the substrate using an additive manufacturing process. The sensors may be applied on the substrate using a sputtering process or cladding process. Preferably, the sensors are printed onto the substrate. The sensors may be printed onto the substrates using any suitable printing processes. The sensors may be printed onto the substrate using a 3D printing process, such as for example 3D screen printing, stereolithography, material jetting or LAM. Preferably, the sensors are printed onto the substrate using a 2D printing process, such as for example screen printing or ink jet printing. This way, the construction may be further simplified. Advantageously, the wound cover can be manufactured on an industrial scale.

Alternatively, the sensors may be embedded into the substrate. The sensors may be fully embedded into the substrate. Alternatively, the sensors may be partially arranged on a surface of the substrate. This way, an especially compact and robust construction can be achieved.

Additionally, it is proposed that the substrate consists of a multitude of interconnected islands, wherein each of the islands defines a bearing area for one of the sensors. An “island” is to be understood as a partial area of the substrate which extends at least as far as the corresponding sensor. The islands may be connected via contact points and arranged as chains. Alternatively, the islands may be connected via bridges. For example, each bridge may connect two neighboring islands, alternatively, the bridges may form a bridge network. This way, costs and material may be saved. Advantageously, a flexibility of the substrate and the sensors may be increased.

Additionally, it is proposed that the control unit is separate to the wound cover. For example, the control unit may be arranged on a carrier which may comprise at least one adhesive element and/or clamp element and/or snap element and/or fastening element for attachment of the carrier to a patient wearing the wound cover. Alternatively, the control unit may be arranged inside of a case unit, which allows for a transport and/or placement of the control unit on any surface. The control unit may be part of a telemedical facility. Preferably, the wound cover system comprises at least one communication unit which transfers data from the sensors to the control unit. Advantageously, the communication unit is partially arranged on the wound cover. The communication unit may comprise at least one electrical wire. Preferably, the communication unit is configured to provide a wireless data transfer, especially in the case of the control unit being arranged inside of the case unit. For example, the communication unit may be configured to transfer the data via Bluetooth, radio, infrared radiation, microwave radiation and/or laser transmission. The communication unit may be partially arranged on the substrate or partially form a component together with the sensors. Especially advantageously, the communication unit is partially arranged on the control unit. For example, a first part of the communication unit, which is arranged on the wound cover, may be an emitter, and a second part of the communication unit, which is arranged on the control unit, may be a receiver. Alternatively, both parts of the communication unit may be emitters and receivers. This way, the construction can be simplified further. Advantageously, an arrangement of the control unit on the wound cover is not required, which allows for an increased comfort for patients wearing the wound cover system. Especially advantageously, a remote wound monitoring can be achieved.

The control unit may forward a signal to a separate warning unit configured to inform medical staff about changes of the wound parameter, the control unit forwarding the signal only when a dangerous change of the wound parameter is detected. Preferably, the wound cover system comprises a user interface unit which comprises the control unit and communicates with the wound cover, preferably the communication unit. Preferably, the user interface unit comprises at least one emitter element configured to emit a warning signal to medical staff in case of dangerous changes of the wound parameter. The emitter element may be a LED and/or a speaker and/or a bell. Preferably, the emitter element is a display, in particular a touch display. Advantageously, the emitter element additionally displays a current value and/or a timeline of the wound parameter. Especially advantageously, the user interface unit comprises at least one input element. For example, the input element could allow for an input of a threshold value which, if exceeded, causes the warning signal to be emitted. This could prevent accidental emitting of warning signals due to, for example, an initiation of a wound healing process. Alternatively or additionally, the input element may allow for an input of a frequency in which the control unit evaluates changes of the wound parameter. The emitter element and the input element may be identical. Alternatively, the user interface unit may comprise input elements which are separate from the emitter element, such as buttons, switches, knobs or additional touch elements. This way, a prompt and reliable reaction to dangerous changes of the wound parameter can be achieved. Advantageously, the wound cover system can be adjusted according to the application.

Each user interface unit may communicate with exactly one wound cover and may either need to be discarded upon discarding of the wound cover or need to be assigned to a new wound cover. Preferably, the user interface unit can communicate with a multitude of wound covers. Advantageously, the user interface unit is a multiple-use item, while the wound cover is a one-use item. Especially advantageously, the user interface unit can communicate with a multitude of wound covers simultaneously. An individual warning signal and/or an individual input element may be assigned to each of the wound covers. Alternatively, the user input unit may comprise a switch element configured to allow for a switching between the different wound covers. This way, an efficiency can be increased. Advantageously, an acquisition of multiple user interface units is not necessary. Especially advantageously, a monitoring network for efficient wound monitoring of a multitude of patients can be achieved.

The evaluation of the sensor data for detecting local changes via the control unit may be achieved using different methods. In the following, possible methods are presented, each of these methods may be used by itself or in combination with any of the other methods. None of these methods are a preferred embodiment of the invention, rather, each of these methods provide their own benefits and may be exchanged with any of the other methods at any time.

To improve a detection of the local changes of the wound parameter, it is proposed that the control unit detects local changes of the wound parameter by evaluating absolute values of the wound parameter. Preferably, the control unit evaluates the absolute values of the wound parameter for each sensor in regular time intervals. The control unit may recognize an exceeding of a threshold value by one of the absolute values as a dangerous change of the wound parameter. The control unit may assign individual threshold values to each of the sensors, which may for example be dependent on a position of the corresponding sensor. This way, an easy and prompt detection of changes of the wound parameter can be achieved.

Alternatively or additionally, the control unit may detect local changes of the wound parameter by evaluating local gradients of the wound parameter. Preferably, the control unit compares the values of the wound parameter for each of the sensors in regular time intervals. The control unit may recognize an exceeding of a threshold value, the threshold value being defined by a difference in the values of neighboring sensors, as a dangerous change of the wound parameter. The control unit may assign different threshold values for individual pairs of neighboring sensors, which could for example be dependent on a position of the pair of neighboring sensors. This way, local changes of the wound parameter can be detected in a precise manner.

Furthermore, it is proposed that the control unit detects local changes of the wound parameter by evaluating temporal changes of the wound parameter. Preferably, the control unit evaluates a timeline of the values of the wound parameter for each of the sensors in regular time intervals. The control unit may recognize an exceeding of a threshold value, the threshold value being defined by an increase and/or decrease of the values of one of the sensors over time, as a dangerous change of the wound parameter. The control unit may assign individual threshold values to each of the sensors, which could for example be dependent on their position. This way, local changes of the wound parameter can be detected promptly and regardless of a base temperature of the respective wound area.

The invention also relates to a method for the application of the wound cover system, wherein the wound cover system is used for a wound monitoring. This way, a wound monitoring and a patient comfort can be improved.

The wound cover system shall not be limited by the disclosed applications and embodiments. The wound cover system may deviate from a disclosed number of elements, components and units to provide any of the disclosed functionalities.

Additional aspects are disclosed in the following description of the figures. The figures show exemplary embodiments of the invention. The figures, specification and claims disclose combinations of features. One skilled in the art may view these features on their own or form other, suitable combinations.

Multiple objects of the same kind are only assigned a single reference sign in the figures.

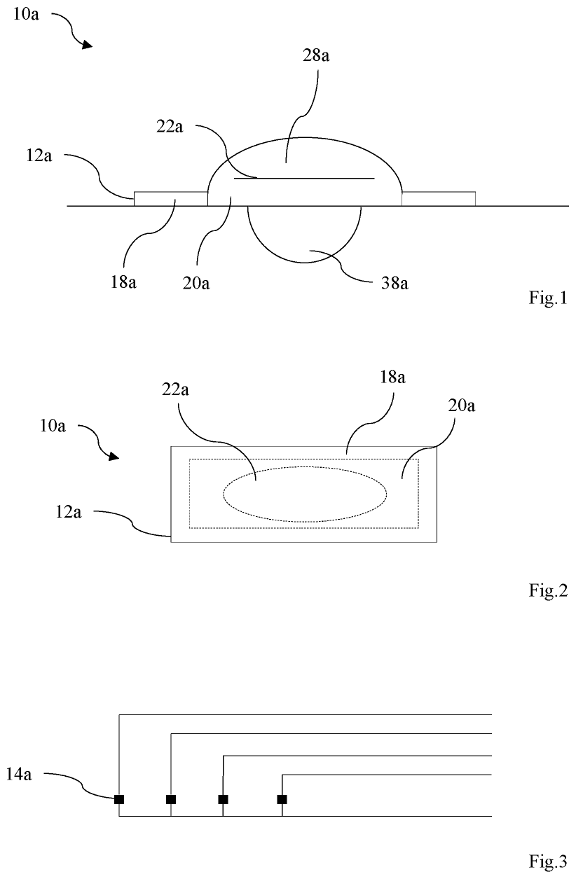

FIGS. 1 and 2 show a wound cover 12a. The wound cover 12a is part of a wound cover system 10a. The wound cover 12a covers a wound 38a. The wound cover 12a comprises an adhesive area 18a. The wound cover 12a comprises a cover area 20a. The adhesive area 18a forms a closed edge around the cover area 20a. The wound 38a is shielded from the outside by the adhesive area 18a. The wound cover 12a comprises a cover material 28a. The cover material 28a consists of cellulose. The cover material 28a is arranged inside of the cover area 20a. The cover material 28a serves, among other things, to absorb fluids exuding from the wound 38a.

The wound cover 12a comprises a substrate 22a. The substrate 22a is a flexible foil. The substrate 22a consists of plastic. The wound cover 12a comprises a multitude of sensors 14a which are spaced apart and configured to measure a wound parameter. The sensors 14a are shown in FIG. 3. The wound parameter is a temperature. The sensors 14a are printed onto the substrate 22a. The sensors 14a and the substrate 22a together form a flexible unit. The sensors 14a are distributed over the cover area 20a. The sensors 14a are distributed over a partial area of the cover area 20a. The partial area forms an oval shape in a top view of the wound cover 12a, as shown in FIG. 2. The partial area is identical to an area covered by the substrate 22a.

The wound cover system 10a comprises a control unit 16a, which is shown in FIG. 4. The control unit 16a detects local changes of the wound parameter based on the data provided by the sensors 14a. The control unit 16a is separate to the wound cover 12a. The wound cover system 10a comprises a user interface unit 26a. The user interface unit 26a comprises the control unit 16a. The wound cover system 10a comprises a communication unit (not shown). The communication unit transfers the data of the sensors 14a to the control unit 16a via Bluetooth. The communication unit comprises a Bluetooth receiver and a Bluetooth emitter. The Bluetooth receiver is arranged on the substrate 22a. The Bluetooth emitter is arranged inside of a case unit 30a. The user interface unit 26a communicates with the wound cover 12a via the communication unit. The user interface unit 26a can communicate with a multitude of further wound covers (not shown), especially when the wound cover 12a is replaced by a new wound cover, which is placed on the wound 38a after a discarding of the wound cover 12a. The user interface unit 26a comprises the case unit 30a. The control unit 16a is arranged inside of the case unit 30a. The user interface unit 26a comprises an input element 32a. The input element 32a is a touch display. The input element 32a allows for an input and output of information. The input element 32a can for example allow for an input of which wound covers 12a the user interface unit 26a needs to communicate with. Furthermore, the input element 32a allows for an emission of a warning signal to medical staff when a dangerous change of the wound parameter is detected by the control unit 16a. Alternatively or additionally, the user interface unit 26a may comprise a separate emitter element configured to display information.

The control unit 16a detects local changes of the wound parameter by evaluating absolute values of the wound parameter. The control unit 16a detects local changes of the wound parameter by evaluating local gradients of the wound parameter. The control unit 16a extracts current values of the wound parameter from the sensor data of each sensor in regular time intervals and compares these current values both with each other and with previous values. Absolute values, differences in values of neighboring sensors 14a as well as temporal changes of the values of individual sensors 14a each are assigned threshold values, and exceeding of any of these threshold values is recognized by the control unit 16a as a dangerous change of the wound parameter and causes the control unit 16a to emit a warning signal to medical staff via the user interface unit 26a.

FIG. 5 shows a schematic process diagram of a method for the application of the wound system 10a. In a covering step 100a, the wound cover 12a is placed over the wound 38a. The adhesive area 18a is pressed onto the surrounding skin to achieve a suitable shielding of the wound 38a from the outside.

In an assignment step 110a, the wound cover 12a is assigned to the user interface unit 36a via the input element 32a. Additionally, the input element 32a may be used to select or adjust threshold values. After the assignment step 110a is finished, the wound cover system 10a provides a constant, local and prompt wound monitoring of the wound 38a. The assignment step 110a follows after the covering step 100a.

In case the control unit 16a detects a dangerous change of the wound parameter, the assignment step 110a is followed by a warning step 120a. The warning step 120a comprises an emitting of a warning signal to medical staff via the input element 32a.

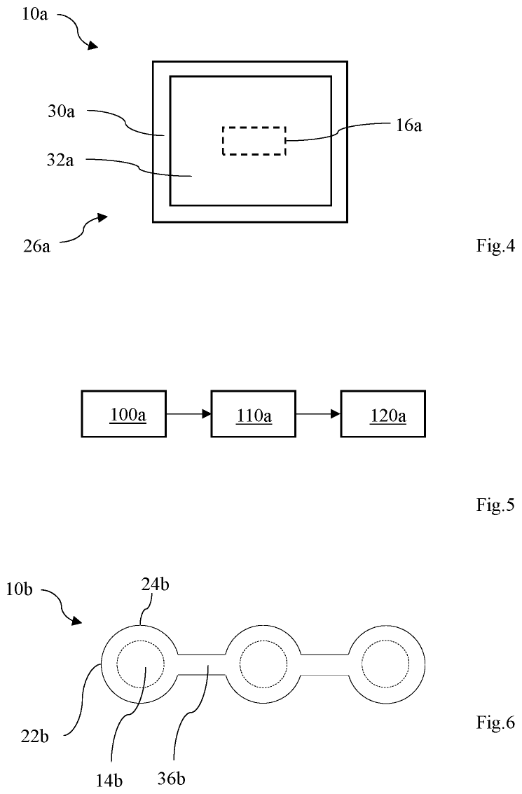

FIG. 6 shows another exemplary embodiment of the wound cover system. The following description only elaborates on differences between the embodiments, wherein components, features and functions that are identical can be found in the description of the embodiments of FIGS. 1 to 5. For the sake of differentiation, the reference signs of the embodiment of FIGS. 1 to 5 are given the suffix a, whilst the reference signs of the embodiment of FIG. 6 are given the suffix b. For identical elements, especially ones that have the same reference sign, FIGS. 1 to 5 and/or the description of the embodiment of FIGS. 1 to 5 may be referred to.

FIG. 6 shows part of a further wound cover system 10b with a substrate 22b and sensors 14b. The substrate 22b is a multi-layer circuit board. The sensors 14b are embedded into the substrate 22b. The substrate 22b consists of a multitude of islands 24b. Each of the islands 24b defines a bearing area for one of the sensors 14b. Neighboring islands 24b are each connected via a bridge 36b.

1. Wound cover system with at least one wound cover comprising a multitude of sensors which are spaced apart and configured to measure at least one wound parameter, and with a control unit which detects local changes of the wound parameter based on the data provided by the sensors.

2. Wound cover system according to claim 1, wherein the wound cover comprises an adhesive area and a cover area, wherein the sensors are distributed over the cover area.

3. Wound cover system according to claim 2, wherein the sensors are distributed over a partial area of the cover area, wherein the partial area forms an oval shape in a top view of the wound cover.

4. Wound cover system according to any of the preceding claims, wherein the wound cover comprises a substrate carrying the sensors, wherein the substrate and the sensors together form a flexible unit.

5. Wound cover system according to claim 4, wherein the sensors are printed onto the substrate.

6. Wound cover system according to claim 4, wherein the sensors are embedded into the substate.

7. Wound cover system according to any of the claims 4 to 6, wherein the substrate consists of a multitude of interconnected islands, wherein each of the islands defines a bearing area for one of the sensors.

8. Wound cover system according to any of the preceding claims, wherein the control unit is separate to the wound cover.

9. Wound cover system according to claim 8, further comprising a user interface unit which comprises the control unit and communicates with the wound cover.

10. Wound cover system according to claim 9, wherein the user interface unit can communicate with a multitude of wound covers.

11. Wound cover system according to any of the preceding claims, wherein the control unit detects local changes of the wound parameter by evaluating absolute values of the wound parameter.

12. Wound cover system according to any of the preceding claims, wherein the control unit detects local changes of the wound parameter by evaluating local gradients of the wound parameter.

13. Wound cover system according to any of the preceding claims, wherein the control unit detects local changes of the wound parameter by evaluating temporal changes of the wound parameter.

14. Method for the application of the wound cover system according to any the preceding claims, wherein the wound cover system is used for a wound monitoring.
